(19) United States
(12) Patent Application Publication
(10) Pub. No.: US 2026/0053471 A1
(43) Pub. Date: Feb. 26, 2026

(54) METHOD, APPARATUS AND ULTRASOUND IMAGING SYSTEM FOR GENERATING THREE-DIMENSIONAL ULTRASOUND MODEL

(71) Applicant: SHENZHEN MINDRAY BIO-MEDICAL ELECTRONICS CO., LTD., Shenzhen (CN)

(72) Inventors: Quan LIU, Shenzhen (CN); Muqing LIN, Shenzhen (CN); Aijun WANG, Shenzhen (CN); Yaoxian ZOU, Shenzhen (CN); Ming ZHANG, Shenzhen (CN)

(73) Assignee: SHENZHEN MINDRAY BIO-MEDICAL ELECTRONICS CO., LTD., Shenzhen (CN)

(21) Appl. No.: 19/310,822

(22) Filed: Aug. 26, 2025

(30) Foreign Application Priority Data
Aug. 26, 2024 (CN) 202411181144.1

(51) Int. Cl. A61B 8/00; A61B 8/08; G06T 7/33; G06T 7/73; G06T 17/00

(52) U.S. Cl. CPC A61B 8/483; A61B 8/085; A61B 8/466; A61B 8/5215; G06T 7/344; G06T 7/75; G06T 17/00; G06T 2207/10132; G06T 2207/30096; G06T 2207/30204; G06T 2210/41

(57) ABSTRACT

The present application discloses a method for generating a 3D ultrasound model, an apparatus therefor, and an ultrasound imaging system. The method includes: obtaining a 2D ultrasound image; obtaining a first target contour corresponding to the structural features of the target portion in the 2D ultrasound image, and determining multiple first deformation reference points on the first target contour; obtaining a base 3D model, and determining a base model section in the base 3D model; obtaining a second target contour corresponding to the structural features of the target portion in the base model section, and determining at least two first deformation control points on the second target contour; determining first model deformation information according to the first deformation reference points and the first deformation control points; and deforming the base 3D model according at least to the first model deformation information to obtain a target 3D model.

### CROSS-REFERENCE TO RELATED APPLICATION

This application claims priority to and the benefit of Chinese Patent Application No. 202411181144.1, filed on Aug. 26, 2024, the disclosure of which is incorporated herein by reference in its entirety.

### TECHNICAL FIELD

The present application relates to the technical field of ultrasound imaging, particularly to methods for generating a three-dimensional (3D) ultrasound model, apparatus for the same, and ultrasound imaging systems.

### BACKGROUND

Compared with other technologies, ultrasound medicine has obvious advantages. As it is non-invasive, it offers the benefits of harmlessness, painlessness, and intuitive use. It is one of the indispensable imaging diagnostic methods in clinical medicine and has been widely used in clinical practice. Traditional ultrasound imaging systems can only provide two-dimensional (2D) ultrasound images of a target portion (a region of interest), making it difficult for patients unfamiliar with ultrasound images, and making it a significant challenge for doctors to describe the patient's condition based on ultrasound images. At the same time, doctors of different qualifications and from different departments have different understandings of ultrasound images, causing unnecessary difficulties in communication among doctors.

3D images or models have the advantages of intuitive image display and wide application in medical teaching and surgical planning. The traditional method for obtaining 3D models of target portions mainly involves using specialized 3D ultrasound probes to acquire 3D ultrasound data of the target portion and then processing the 3D ultrasound data to obtain the 3D model of the target portion. However, the ultrasound probes used in this method are expensive and have a narrow imaging field of view, or the resolution of the 3D ultrasound data obtained by these ultrasound probes is typically lower than that of 2D ultrasound data, resulting in insufficient resolution of the 3D models synthesized by this imaging method and ultimately limiting the application scenarios of this method.

### SUMMARY

The present application provides a methods, apparatus and ultrasound imaging systems for generating a 3D ultrasound model.

According to a first aspect of the present disclosure, a method for generating a 3D ultrasound model provided in some embodiments may include:
obtaining a 2D ultrasound image corresponding to a target ultrasound section of a target portion, the 2D ultrasound image containing structural features of the target portion and lesion features of the target portion;
obtain a first target contour corresponding to the structural features of the target portion and a first lesion contour corresponding to the lesion features of the target portion in the 2D ultrasound image, and determining a plurality of first deformation reference points on the first target contour and a plurality of second deformation reference points on the first lesion contour;
obtaining a base 3D model corresponding to the target portion, and determining a base model section matching the target ultrasound section in the base 3D model, wherein the base model section contains the structural features of the target portion and the lesion features of the target portion;
obtaining a second target contour corresponding to the structural features of the target portion in the base model section, and determining on the second target contour at least two first deformation control points corresponding to at least two of the plurality of first deformation reference points;
obtaining a second lesion contour corresponding to the lesion features of the target portion in the base model section, and determining on the second lesion contour at least two second deformation control points corresponding to at least two of the plurality of second deformation reference points;
determining first model deformation information of the base 3D model based on the first deformation reference points and the first deformation control points;
determining second model deformation information of the base 3D model based on the second deformation reference points and the second deformation control points; and
deforming the base 3D model based at least on the first model deformation information and the second model deformation information, thereby obtaining a target 3D model.

From the above technical solution provided in this application, it can be seen that the method for generating a 3D ultrasound model provided herein obtains a 2D ultrasound image of the target portion with actual morphology, and from the 2D ultrasound image, obtains the first target contour corresponding to the structural features of the target portion and the first lesion contour corresponding to the lesion features of the target portion in the 2D ultrasound image; and accordingly, by utilizing the resulted first target contour as a reference target of the second target contour for the structural features of the target portion in the base model section associated with the base 3D model, and by utilizing the resulted first lesion contour as a reference target of the second lesion contour for the lesion features of the target portion in the base model section associated with the base 3D model, deformation constraints are imposed on the structural features and lesion features of the target portion in the base 3D model, thereby making the deformed base 3D model more resemble the real morphological model of the target portion, that is, the 3D model of the target portion finally obtained is more accurate and realistic.

According to a second aspect of the present disclosure, a method for generating a 3D ultrasound model provided in some embodiments may include:
obtaining a 2D ultrasound image corresponding to the target ultrasound section of the target portion, the 2D ultrasound image at least containing structural features of the target portion;
obtaining a first target contour corresponding to the structural features of the target portion in the 2D ultrasound image, and determining a plurality of first deformation reference points on the first target contour;
obtaining a base 3D model corresponding to the target portion, and determining a base model section matching the target ultrasound section in the base 3D model, the base model section containing structural features of the target portion;
obtaining a second target contour corresponding to the structural features of the target portion in the base model section, and determining on the second target contour at least two first deformation control points corresponding to at least two of the plurality of first deformation reference points;
determining first model deformation information of the base 3D model based on the first deformation reference point and the first deformation control point; and
deforming the base 3D model at least based on the first model deformation information, thereby obtaining a target 3D model.

From the above technical solution provided in this application, it can be seen that the method for generating a 3D ultrasound model provided by herein obtains 2D ultrasound images of the target portion with actual morphology and obtains the first target contour corresponding to the structural features of the target portion in the 2D ultrasound image; and accordingly, by utilizing the resulted first target contour as a reference target for the structural features of the target portion in the base model section associated with the base 3D model, deformation constraints are imposed on the structural features of the target portion in the base 3D model. This makes the deformed base 3D model more closely resemble the real morphological model of the target portion, that is, the 3D model of the target portion finally obtained is more accurate and realistic.

According to a third aspect of the present disclosure, an apparatus for generating a 3D ultrasound model provided in some embodiments may include:
a memory for storing programs; and
a processor for implementing the steps of the method for generating a 3D ultrasound model as described in any one of the foregoing embodiments by executing the programs stored in the memory.

According to a fourth aspect of the present disclosure, an ultrasound imaging system provided in some embodiments may include:
an ultrasound probe including a plurality of transducer elements;
a transmit circuit for exciting the ultrasound probe to emit ultrasound waves to a target portion;
a receive circuit for controlling the ultrasound probe to receive echo signals of the ultrasound waves;
a memory for storing programs;
a processor for generating 2D ultrasound image corresponding to the target ultrasound section of the target portion based on the echo signals, and further for implementing the steps of the method for generating a 3D ultrasound model as described above by executing the programs stored in the memory; and
a display at least for displaying the ultrasound image and/or the 3D model that are generated by the processor.

It should be understood that the above general description and the detailed description to follow are merely exemplary and explanatory and are not intended to limit the present application.

### BRIEF DESCRIPTION OF THE DRAWINGS

To more clearly illustrate the technical solutions of the embodiments of the present application, the drawings that need to be used in the description of the embodiments will be briefly introduced below. Obviously, the drawings described below are some embodiments of the present application. For those skilled in the art, other drawings can be obtained without creative labor based on these drawings.

FIG. 1 illustrates a block diagram of the structure of an ultrasound imaging system according to an embodiment of the present application;

FIG. 2 illustrates a schematic flowchart of a method for generating a 3D ultrasound model according to an embodiment of the present application;

FIG. 3 illustrates a 2D ultrasound image, obtained by the ultrasound imaging system, of a target portion in the long-axis plane;

FIG. 4 illustrates a 2D ultrasound image, obtained by the ultrasound imaging system, of a target portion in the short-axis plane;

FIG. 5 illustrates a schematic diagram of obtaining the first target contour of the target portion, as well as the first deformation reference points on the first target contour from the 2D ultrasound image;

FIG. 6 illustrates a schematic diagram of obtaining the second target contour of the target portion, as well as the first deformation control points on the second target contour from the base model section;

FIG. 7 illustrates a schematic diagram of obtaining the target 3D model by deforming the base 3D model according to a first deformation magnitude;

FIG. 8 illustrates a schematic diagram of obtaining the first target contour of the target portion, the first deformation reference points on the first target contour, the first lesion contour, and the second deformation reference points on the first lesion contour from the 2D ultrasound image;

FIG. 9 illustrates a schematic diagram of obtaining the second target contour of the target portion, the first deformation control points on the second target contour, the second lesion contour, and the second deformation control points on the second lesion contour from the base model section;

FIG. 10 illustrates a schematic diagram of obtaining the target 3D model by deforming the base 3D model according to a first deformation magnitude and a second deformation magnitude;

FIG. 11 illustrates a schematic diagram of displaying, on the target 3D model, the data obtained by measuring the 2D ultrasound image of the target portion; and

FIG. 12 illustrates a schematic diagram of fusing and displaying the 2D ultrasound image of the target portion and the target 3D model.

### DETAILED DESCRIPTION

The following describes the technical solutions in the embodiments of the present application clearly and completely with reference to the accompanying drawings. Clearly, the described embodiments are only part of the embodiments of the present application, but not all embodiments. All other embodiments obtained by those skilled in the art based on the embodiments of the present application without creative exercise shall fall within the scope of protection of the present application.

The flowcharts in the accompanying drawings are illustrative only. They neither necessarily include all contents and operations/steps, nor must be executed strictly in the described sequence. For example, certain operations/steps may be decomposed, combined or partially merged. Consequently, the actual execution order may vary depending on specific contexts.

It should be understood that the terms used herein in the specification of this application serves solely to describe particular embodiments and are not intended to restrict the present application. As used in this specification and the appended claims, unless the context clearly dictates otherwise, the singular forms “a”, “an” and “the” encompass plural referents.

The following describes in detail some embodiments of the present application with reference to the accompanying drawings. Absent conflict, the embodiments described herein and features therein may be combined with each other.

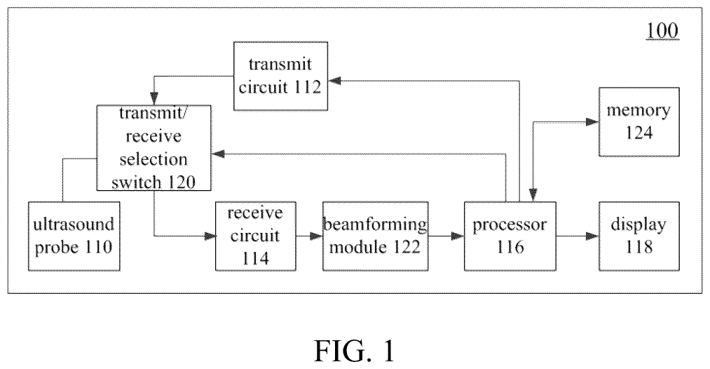

Please refer to FIG. 1. FIG. 1 depicts a schematic block diagram of an ultrasound imaging system according to an embodiment of the present disclosure.

As shown in FIG. 1, the ultrasound imaging system 100 includes an ultrasound probe 110, a transmit circuit 112, a receive circuit 114, a processor 116, and a display 118. Further, the ultrasound imaging system may also include a transmit/receive selection switch 120 and a beamforming module 122. The transmit circuit 112 and the receive circuit 114 may be connected to the ultrasound probe 110 via the transmit/receive selection switch 120.

The ultrasound probe 110 includes a plurality of transducer elements (referred to as elements). These transducer elements may be arranged in a row to form a linear array, or arranged in a 2D matrix to form a planar array. The transducer elements may also form a convex array. For example, the transducer elements may be arranged in a single row along a predefined predefined direction, or arranged in multiple rows along a predefined direction.

The transducer elements are configured to emit ultrasound waves in response to the excitation electrical signal or convert the received ultrasound waves into electrical signals. Therefore, each transducer element can be used to achieve the two-way conversion between electrical pulse signals and ultrasound waves, thereby enabling the emission of ultrasound waves to a target portion of tissue of an object under examination and also the reception of ultrasound wave echoes reflected back by the tissue. During ultrasound testing, the emission sequence and reception sequence can be controlled to determine which transducer elements are used for emitting ultrasound waves and which are used for receiving ultrasound echoes, or to control the transducer elements to operate in time-division multiplexing mode for emitting ultrasound waves or receiving ultrasound echoes. The transducer elements participating in the emission of ultrasound waves can be simultaneously excited by electrical signals to emit ultrasound waves simultaneously; or, the transducer elements participating in the emission of the ultrasound beam can also be excited by several electrical signals with defined time intervals, thereby emitting ultrasound waves in a sequence of pulses separated by specific time intervals.

During the ultrasound imaging process, the processor 116 controls the transmit circuit 112 to send the transmitting pulses (which have been delayed and focused) via the transmit/receive selection switch 120 to the ultrasound probe 110. The ultrasound probe 110 is excited by the transmitting pulses and emits an ultrasound beam towards the target portion. After a certain delay, it receives the ultrasound echoes reflected back from the target portion and converts them back to electrical signals. The receive circuit 114 receives the electrical signals from the ultrasound probe 110 (representing the ultrasound echo signals) and sends these ultrasound echo signals to the beamforming module 122. The beamforming module 122 performs focusing delay, weighting, and channel summation processing on the ultrasound echo data, and then passes the processed echo data to the processor 116. The processor 116 processes the (beamformed) ultrasound echo signals through signal detection, signal enhancement, data conversion, and logarithmic compression to form an ultrasound image. The ultrasound image obtained by the processor 116 can be displayed on the display 118 or stored in the memory 124.

Optionally, the processor 116 may be implemented as software, hardware, firmware, or any combination thereof, and may be realized using a single or multiple application-specific integrated circuits (ASICs), a single or multiple general-purpose integrated circuits, a single or multiple microprocessors, a single or multiple programmable logic devices, or any combination of the circuits and/or devices listed above, or other suitable circuits or devices. Moreover, the processor 116 can control other components in the ultrasound imaging system 100 to perform the corresponding steps of the methods as described in various embodiments herein.

The display 118 is connected to the processor 116. The display 118 can be a touchscreen display, a liquid crystal display (LCD), or the like. Alternatively, the display 118 can be an external display device, such as a standalone LCD monitor or a television set, separate from the ultrasound imaging system 100. As another options, the display 118 could be the screen of a portable electronic device, such as a smartphone or a tablet computer. Furthermore, there may be one or more displays 118.

The display 118 can show the ultrasound images generated by the processor 116. Additionally, while displaying the ultrasound images, the display 118 can also provide a graphical interface for human-machine interaction (HMI) to the user. One or more controls can be set on the graphical interface, allowing the user to input operation instructions through a HMI device to control these controls and thereby perform corresponding control operations. For instance, icons can be displayed on the graphical interface, and the user can manipulate an icon on the HMI device to execute specific functions, such as drawing a region of interest (ROI) box on the ultrasound image.

Optionally, the ultrasound imaging system 100 may include additional HMI devices beyond the display 118, which are connected to the processor 116. For example, these devices connect to the processor 116 via an external input/output (I/O) port, which may utilize a wireless communication module, a wired communication module, or a combination of both. The external I/O port may be implemented based on USB, bus protocols (e.g., CAN), and/or wired network protocols.

The HMI device can include an input device for detecting user input information. The input information can be, for example, control instructions for the ultrasound wave transmission/reception timing, operation input instructions for drawing points, lines, or boxes on the ultrasound image, or other types of instructions. The input device can include one or a combination of a keyboard, mouse, scroll wheel, trackball, mobile input device (such as a touchscreen device, a mobile phone, etc.), multi-functional knob. The HMI device can also include output devices such as a printer.

The ultrasound imaging system 100 may also include a memory 124 for storing instructions executed by the processor, received ultrasound echoes, ultrasound images, etc. The memory can be a flash memory card, solid-state memory, hard disk, etc. It can be volatile memory and/or non-volatile memory, removable memory and/or non-removable memory, etc. For example, the memory 124 can be a plug-in hard drive, Smart Media Card (SMC), Secure Digital (SD) card, flash memory card in the ultrasound imaging system 100.

It should be understood that the components included in the ultrasound imaging system 100 shown in FIG. 1 are merely illustrative; the system may include more or fewer components, which are not limited by the present disclosure.

In some embodiments, the ultrasound imaging system 100 is also provided for performing the steps of a method for generating a 3D ultrasound model. Specifically, the transmit circuit 112 of the ultrasound imaging system 100 is configured to excite the ultrasound probe 110 to emit ultrasound waves to the target portion; the receive circuit 114 of the ultrasound imaging system 100 is configured to control the ultrasound probe 110 to receive the echoes of the ultrasound waves to obtain an echo signal of the ultrasound waves; the processor 116 of the ultrasound imaging system 100 is configured to generate a 2D ultrasound image of the target portion based on the echo signal, and is also configured to perform the steps of the method for generating a 3D ultrasound model as described in any embodiment or example of the present application to obtain a corresponding 3D model; and the display 118 is configured to display the 2D ultrasound image and/or the 3D model.

The above only describes the main functions of each component of the ultrasound imaging system. For more details, please refer to the relevant descriptions of the method for generating a 3D ultrasound model in the following text; details will not be repeated here.

In some embodiments, an apparatus for generating a 3D ultrasound model is also provide in some embodiments of the present application for implementing the method for generating a 3D ultrasound model mentioned above. Exemplarily, the apparatus for generating a 3D ultrasound model includes a memory and a processor, wherein the memory is configured to store programs, and the processor is configured to implement the steps of the method for generating a 3D ultrasound model described in any embodiment or example of the present application by executing the programs stored in the memory.

It is to be understood that, the apparatus for generating a 3D ultrasound model may be configured to share the memory and/or the processor with the ultrasound imaging system 100; or, the memory and the processor of the apparatus for generating a 3D ultrasound model are independent of the ultrasound imaging system 100, that is, the apparatus for generating a 3D ultrasound model does not share the memory and the processor with the ultrasound imaging system 100. It is only necessary that the processor of the apparatus for generating a 3D ultrasound model is configured to perform the steps of the method for generating a 3D ultrasound model as described in any embodiment or example of the present application by executing the program stored in the memory.

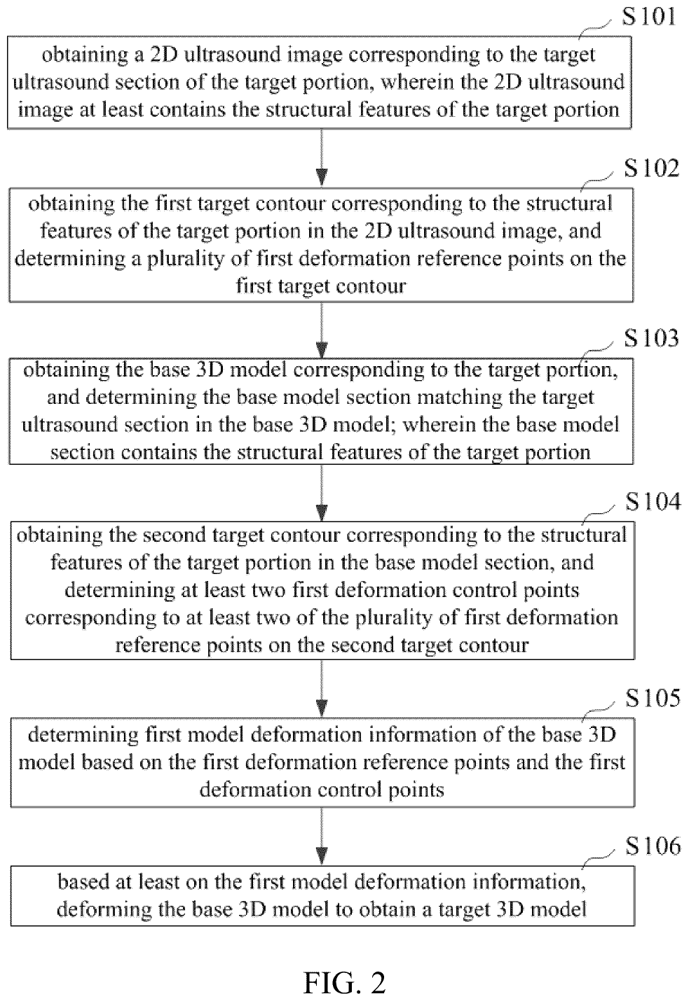

Please refer to FIG. 2. FIG. 2 is a schematic flowchart of a method for generating a 3D ultrasound model according to an embodiment of the present disclosure.

As shown in FIG. 2, the method for generating a 3D ultrasound model may be implemented in the aforementioned ultrasound imaging system 100 and executed by the processor thereof, or it may be implemented in the aforementioned apparatus for generating a 3D ultrasound model and executed by the processor thereof. In this application, the method for generating a 3D ultrasound model being applied to the ultrasound imaging system 100 is described by way of example. The method for generating a 3D ultrasound model comprises steps S101 to S106.

Step S101: obtaining a 2D ultrasound image corresponding to the target ultrasound section of the target portion, wherein the 2D ultrasound image at least contains the structural features of the target portion.

By way of example, the target portion is defined as the target tissue for which a 2D ultrasound image is to be generated. The target portion includes, without limitation, areas such as the patient's neck, chest, abdomen, pelvic organs, and peripheral vessels. It is to be understood that the target portion in the neck may include organs such as the thyroid, parotid gland, submandibular gland, and neck vessels. In the chest, the target portion may include structures such as the pleura, lung, heart along with surrounding large blood vessels, and mediastinal lymph nodes. For the abdominal cavity, the target portion may include organs such as the liver, gallbladder, pancreas, spleen, kidney, ureter, bladder, uterus, ovary, fallopian tube, and prostate.

When obtaining the 2D ultrasound image corresponding to the target ultrasound section of the target portion of a patient, the ultrasound probe 110 emits ultrasound waves toward the target portion, generating at least one frame of said image from the received ultrasound echo signals. The 2D ultrasound image contains at least the structural features of the target portion.

It should be understood that the structural features describe the structural attributes of the target portion and differentiate the type of the target portion. In medicine, different target portions exhibit different structural features; for example, the heart has structural features unique to cardiac tissue, while the uterus has structural features unique to uterine tissue, thereby enabling differentiation of the target portion.

Optionally, to obtain the 2D ultrasound image corresponding to the target ultrasound section of the target portion, at least two frames of 2D ultrasound images corresponding to at least two different long-axis planes of the target portion may be obtained, each containing the structural features of the target portion. Here, any two such different long-axis planes are disposed at an angle to each other. For example, the angle between any two different long-axis planes may range from 30° to 90°, i.e., it may be 30°, 40°, 50°, 60°, 70°, 80°, or 90°.

That is, the ultrasound probe 110 emits ultrasound waves toward the target ultrasound section of the target portion on at least two different long-axis planes of the target portion, and acquires, based on the received ultrasound echo signals, at least two frames of 2D ultrasound images corresponding to the at least two different long-axis planes of the target portion. The two different long-axis planes are disposed at an angle to each other, for example, 90°.

Optionally, the 2D ultrasound image corresponding to the target ultrasound section of the target portion may also be obtained by acquiring at least one frame of a 2D ultrasound image corresponding to at least one long-axis plane and at least one frame of a 2D ultrasound image corresponding to at least one short-axis plane of the target portion, each containing the structural features of the target portion.

That is, the ultrasound probe 110 emits ultrasound waves toward the target portion on at least one long-axis plane, and based on the received ultrasound echo signals, obtains at least one frame of a 2D ultrasound image corresponding to said at least one long-axis plane; and emits ultrasound waves toward the target portion on at least one short-axis plane of the target portion and, based on the received ultrasound echo signals, obtains at least one frame of a 2D ultrasound image corresponding to said at least one short-axis plane.

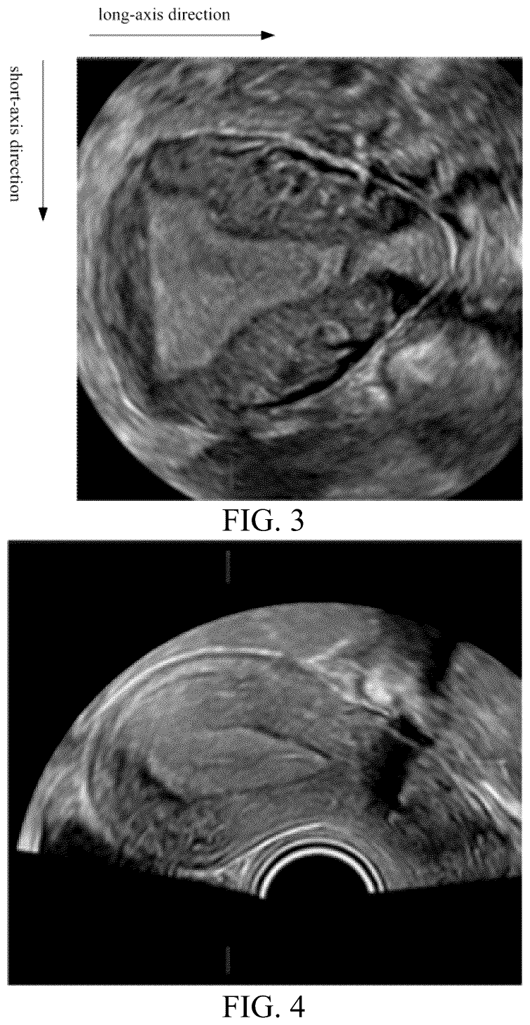

As shown in FIGS. 3 and 4, FIG. 3 is a 2D ultrasound image of the target portion in the long-axis plane obtained by the ultrasound imaging system 100, wherein the 2D ultrasound image includes the structural features of the target portion; while FIG. 4 is a 2D ultrasound image of the target portion in the short-axis plane obtained by the ultrasound imaging system 100, wherein the 2D ultrasound image includes the structural features of the target portion.

Step S102: obtaining the first target contour corresponding to the structural features of the target portion in the 2D ultrasound image, and determining a plurality of first deformation reference points on the first target contour.

For example, the method for identifying a corresponding contour in the 2D ultrasound image may involve: (a) manually delineation of the contour to be identified; (b) processing the image through a pre-trained contour recognition model to output the corresponding contour; or (c) applying a predefined contour recognition algorithm to detect the corresponding contour.

After obtaining the first target contour corresponding to the structural features of the target portion, a plurality of first deformation reference points are determined on the first target contour. The first deformation reference points may be determined via manual user selection or by automatic generation via a predefined algorithm on the first target contour.

In some embodiments, the first target contour corresponding to the structural features of the target portion in the 2D ultrasound image may be obtained by: in response to a first contour delineation operation performed by a user on the 2D ultrasound image, determining the first target contour corresponding to the structural features of the target portion from the 2D ultrasound image.

For example, the obtained 2D ultrasound image of the target portion is displayable on a display. A user may visually identify the structural contour corresponding to the structural features of the target portion from the displayed 2D ultrasound image on the display based on experience, and subsequently manipulate an input device to perform the first contour delineation operation on the 2D ultrasound image. This operation enables delineation of the first target contour corresponding to the structural features of the target portion from the 2D ultrasound image. Accordingly, the ultrasound imaging system 100, in response to the first contour delineation operation performed on the 2D ultrasound image, and determines the first target contour corresponding to the structural features of the target portion from the 2D ultrasound image. It is to be understood that the input device includes, but is not limited to, at least one of: a keyboard, a mouse, a touch panel, a stylus, and a key.

For example, when the input device comprises a mouse or a stylus, a user may visually identify a structural contour corresponding to the structural features of the target portion in the 2D ultrasound image displayed on the display, and manipulate the mouse or stylus to execute a contour delineation operation on the 2D ultrasound image thereby enabling the ultrasound imaging system 100 to generate the first target contour corresponding to the structural features of the target portion; wherein the ultrasound imaging system 100 can obtain the first target contour corresponding to the structural features of the target portion from the 2D ultrasound image by processing the contour delineation operation performed by the user through the input device.

In some embodiments, the first target contour corresponding to the structural features of the target portion in the 2D ultrasound image may also be obtained by: determining a plurality of first contour control points on the 2D ultrasound image, and interpolating between each pair of adjacent first contour control points to form a first contour curve (also known as interpolation algorithm), and defining the first contour curve as the first target contour corresponding to the structural features of the target portion.

For example, the obtained 2D ultrasound image of the target portion can be displayed on a display, and a user can identify the structural contour corresponding to the structural features of the target portion from the 2D ultrasound image displayed on the display based on experience, and determine a plurality of first contour control points in the structural contour corresponding to the structural features of the target portion through an input device. After the ultrasound imaging system 100 recognizes the plurality of first contour control points determined by the user, it forms a first contour curve between each pair of adjacent first contour control points through a predefined interpolation algorithm, and takes the first contour curve as the first target contour corresponding to the structural features of the target portion. It is to be understood that the interpolation algorithm includes but is not limited to the B-spline interpolation algorithm, also known as the B-Spline interpolation method.

In some embodiments, the first target contour corresponding to the structural features of the target portion in the 2D ultrasound image can also be obtained from the 2D ultrasound image by utilizing a predefined first contour recognition model.

For example, a pre-trained first contour recognition model is used for performing contour recognition on the 2D ultrasound image, thereby obtaining the first target contour corresponding to the structural features of the target portion. The first contour recognition model can be trained using 2D ultrasound images with the structural features of the target portion as training data.

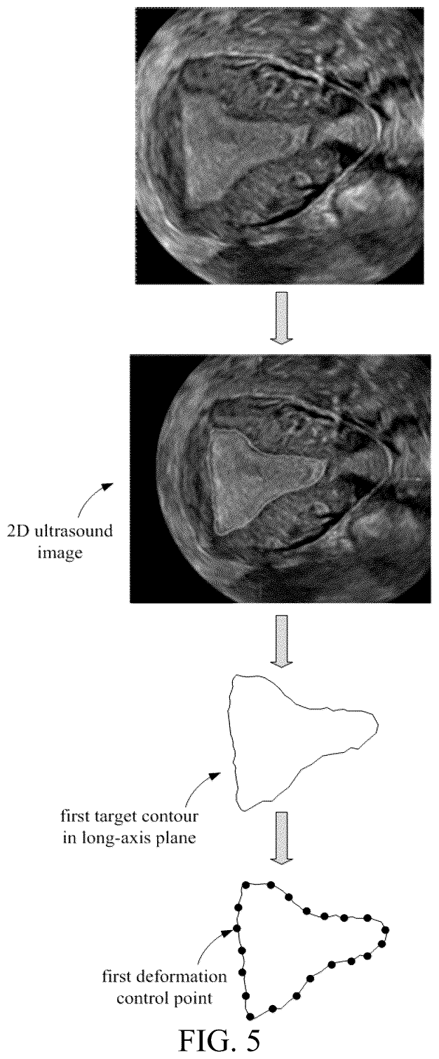

As shown in FIG. 5, the ultrasound imaging system 100 presents a 2D ultrasound image of the target portion in the long-axis plane, and obtains the first target contour corresponding to the structural features of the target portion in the 2D ultrasound image based on a user manual selection, a predefined algorithm, or a model recognition.

In some embodiments, a plurality of first deformation reference points are determined on the first target contour, by: a predefined first rule, or a manual user selection operation.

For example, the first rule comprises at least one of: (a) distributing a random or predefined number of first deformation reference points on the first target contour wherein adjacent first deformation reference points remain equidistant; (b) distributing a random or predefined number of first deformation reference points on the first target contour wherein the distance between adjacent first deformation reference points varies stochastically; (c) distributing a predefined number of first deformation reference points on the first target contour wherein the spacing distribution between adjacent first deformation reference points correlates with the type of the target portion.

In some embodiments, a plurality of first deformation reference points are determined on the first target contour by: (a) determining a plurality of first key reference points on the first target contour, determining first key contour points on the contour between each adjacent pair of the first key reference points based on a predefined second rule, and defining the combined set of first key contour points and the first key reference points as the plurality of first deformation reference points on the first target contour.

For example, the user positions multiple first key reference points on the first target contour based on experience; or, the ultrasound imaging system 100 automatically determines the first key reference points using a predefined deep learning algorithm or deep learning model. The first key reference points are then used to divide the first target contour corresponding to the structural features of the target portion into multiple first sub-contours. On each first sub-contour: the multiple first key contour points are defined, and the combined set of first key contour points and first key reference points constitute the plurality of first deformation reference points on the first target contour. Optionally, adjacent first key contour points on each first sub-contour maintain uniform spacing.

As shown in FIG. 5, the plurality of first deformation reference points are determined on the first target contour obtained from the 2D ultrasound image corresponding to the long-axis plane.

Step S103: obtaining the base 3D model corresponding to the target portion, and determining the base model section matching the target ultrasound section in the base 3D model; wherein the base model section contains the structural features of the target portion.

For example, there are multiple approaches to obtain the base 3D model corresponding to the target portion. For instance, it can be achieved by collecting the 3D ultrasound volume data of the target portion of a patient and converting the 3D ultrasound volume data of the target portion to a 3D mesh model using relevant algorithms as the 3D template model; alternatively, by collecting CT (Computed Tomography) data of the target portion of a patient and converting the CT data of the target portion to a 3D mesh model via related algorithms as the 3D template model; or by constructing the 3D template model of the target portion through 3D modeling techniques. Such 3D template models may be 3D mesh models with vertices uniformly distributed across the model. Optionally, each face of the 3D mesh model is filled with a patch, and texture information may be mapped onto the patch.

The base 3D model comprises at least the structural features corresponding to the target portion. Given that the structural features of the target portion vary among patients, the base 3D model is adjustable to better approximate the current target patient under examination, thereby achieving a more accurate and anatomically faithful 3D model corresponding to the ultrasound image of the target portion.

In this embodiment, after the base 3D model is obtained, a matching base model section corresponding to the target ultrasound section is determined within the base 3D model. For example, if the target ultrasound section is a section of the target portion in the long-axis direction at a predefined angle, then a section in the long-axis direction at the same predefined angle is determined in the base 3D model corresponding to the target portion. This makes the base model section obtained from the base 3D model substantially correspond to the same section of the target portion as the target ultrasound section, thus effectively enhancing the accuracy of subsequent deformation. Furthermore, the base model section obtained from the base 3D model contains the structural features of the target portion.

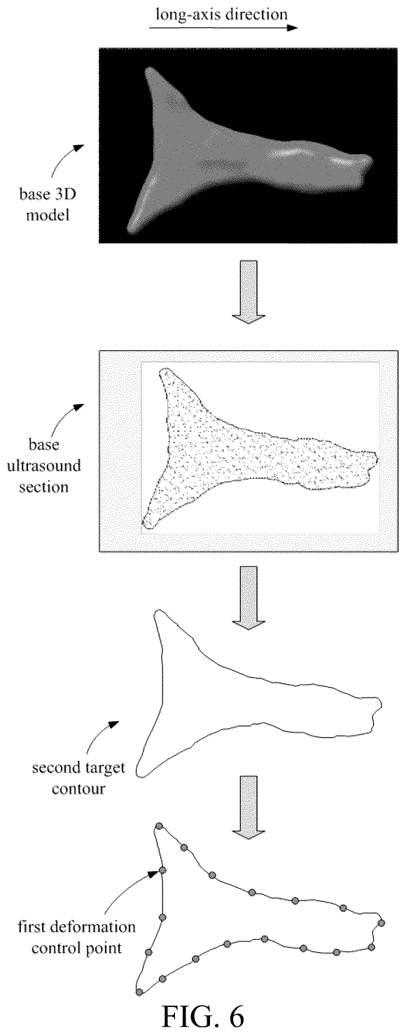

As shown in FIG. 6, at the predefined angle corresponding to the base 3D model, the base model section of the target portion in the long-axis direction can be obtained.

Step S104: obtaining the second target contour corresponding to the structural features of the target portion in the base model section, and determining at least two first deformation control points corresponding to at least two of the plurality of first deformation reference points on the second target contour.

For example, the way to obtain the second target contour corresponding to the structural features of the target portion in the base model section can be to manually draw the contour of the contour to be identified corresponding to the base model section, or to use a pre-trained contour recognition model, taking the base model section as the input of the contour recognition model to obtain the corresponding contour in the base model section, or to use a predefined contour recognition algorithm to recognize the corresponding contour in the base model section, or to use an edge recognition algorithm to recognize the corresponding contour in the base model section.

After the second target contour corresponding to the structural features of the target portion is obtained, multiple first deformation control points are determined on the second target contour. The first deformation control points may be determined by manual selection by a user or be generated automatically on the second target contour through a predefined algorithm.

Optionally, the second target contour corresponding to the structural features of the target portion in the base model section may be obtained by: in response to the user's third contour delineation operation on the base model section, determining, from the base model section, the second target contour associated with the structural features of the target portion.

For example, the base model section of the target portion can be displayed on a display. A user, based on experience, can identify the structural contour corresponding to the structural features of the target portion from the base model section displayed on the display. The user can then, by manipulating the input device, perform a third contour delineation operation on the base model section to delineate the second target contour corresponding to the structural features of the target portion. The ultrasound imaging system 100 responds to the user's third contour delineation operation on the base model section and determines the second target contour corresponding to the structural features of the target portion from the base model section. It is to be understood that the input device includes at least one of a keyboard, a mouse, a touch panel, a stylus and a key.

Optionally, the second target contour corresponding to the structural features of the target portion in the base model section may be obtained by: determining multiple third contour control points on the base model section, and interpolating between each pair of adjacent third contour control points through interpolation (also known as interpolation algorithm) to form a third contour curve, and defining the third contour curve as the second target contour corresponding to the structural features of the target portion.

For example, the base model section of the target portion obtained can be displayed on the display; a user, based on experience, can identify the structural contour corresponding to the structural features of the target portion from the base model section displayed on the display, and determine multiple third contour control points in the structural contour corresponding to the structural features of the target portion via the input device. After the ultrasound imaging system 100 recognizes the multiple third contour control points determined by the user, it forms a third contour curve between each pair of adjacent third contour control points through interpolation based on a predefined interpolation algorithm, and takes the third contour curve as the second target contour corresponding to the structural features of the target portion. It is to be understood that the interpolation algorithm includes, but is not limited to, the B-spline interpolation algorithm, also referred to as the B-Spline interpolation method.

Optionally, a way to obtain the second target contour corresponding to the structural features of the target portion in the base model section may be: obtaining it from the base model section through a predefined third contour recognition model.

For example, the pre-trained third contour recognition model is used to perform contour recognition on the base model section, thereby obtaining the second target contour corresponding to the structural features of the target portion. The third contour recognition model can be trained using images with the structural features of the target portion as training data.

Optionally, obtaining the second target contour corresponding to the structural features of the target portion in the base model section can be achieved by: identifying the edges of the structural features of the target portion in the base model section through a predefined edge recognition algorithm, thereby obtaining the second target contour corresponding to the structural features of the target portion.

After obtaining the second target contour corresponding to the structural features of the target portion in the base model section, at least two first deformation control points corresponding to at least two of the multiple first deformation reference points are determined on the second target contour. Optionally, the first deformation control points on the second target contour and the first deformation reference points on the first target contour correspond one-to-one.

For example, given that the same target portion has substantially identical basic structural features, the corresponding first deformation control point can therefore be determined on the second target contour at the second position corresponding to the first position (of the first deformation reference point) on the first target contour.

During the deformation process for the structural features of the base 3D model, it may only be necessary to deform a subset of the first deformation control points. Therefore, to ensure the deformation effect, it is necessary to determine, on the second target contour, at least two first deformation control points corresponding to at least two of the multiple first deformation reference points need. The second target contour can then be deformed based on the corresponding deformation control points and deformation reference points.

Step S105: determining first model deformation information of the base 3D model based on the first deformation reference points and the first deformation control points.

Optionally, determining the first model deformation information of the base 3D model based on the first deformation reference point and the first deformation control point includes: determining multiple first deformation magnitudes between the multiple first deformation reference points on the first target contour and their corresponding first deformation control points on the second target contour; and determining the first model deformation information of the base 3D model based at least on the multiple first deformation magnitudes.

For example, the first deformation control points on the second target contour can be in one-to-one correspondence with the first deformation reference points on the first target contour, thereby establishing a first mapping relationship (also called a first deformation magnitude) between the first deformation control points on the second target contour and the first deformation reference points on the first target contour. Or, one first deformation control point on the second target contour can correspond to multiple first deformation reference points on the first target contour, thereby establishing the first mapping relationship (also called the first deformation magnitude) between the first deformation control points on the second target contour and the first deformation reference points on the first target contour. Or, multiple first deformation control points on the second target contour can correspond to one first deformation reference point on the first target contour, thereby establishing the first mapping relationship (also called the first deformation magnitude) between the first deformation control points on the second target contour and the first deformation reference points on the first target contour. Thus, the first model deformation information of the base 3D model can be obtained based on the multiple first deformation magnitudes corresponding to the associations between the multiple first deformation reference points and multiple first deformation control points.

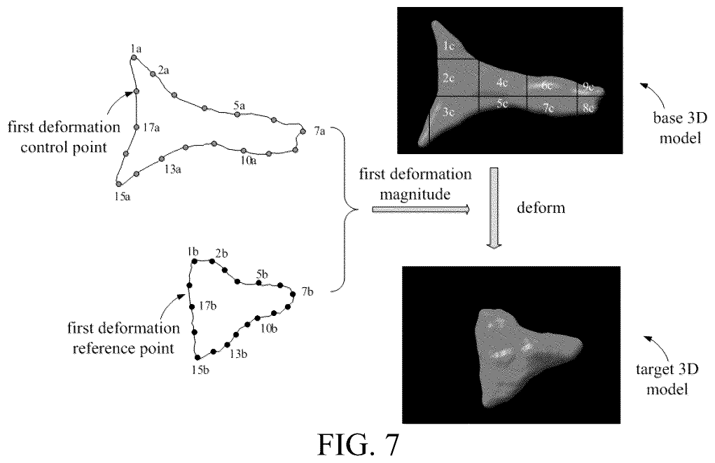

As shown in FIG. 7, for instance, there are 18 first deformation reference points (numbered 1a to 18a respectively) on the first target contour, and there are also 18 first deformation control points (numbered 1b to 18b respectively) on the second target contour. In this configuration, a one-to-one correspondence is established between these points such that point 1a corresponds to point 1b, point 2a corresponds to point 2b, point 3a corresponds to point 3b, and so forth, continuing sequentially to point 17a corresponding to point 17b and point 18a corresponding to point 18b. Each pair of corresponding points has an associated first deformation magnitude. The first deformation magnitudes associated with each of these 18 correspondences are used as the first model deformation information of the base 3D model.

Optionally, determining the first model deformation information of the base 3D model based on the first deformation reference points and the first deformation control points includes: determining multiple first deformation magnitudes corresponding to associations between: (i) multiple first deformation reference points on the first target contour, and (ii) multiple first deformation control points on the second target contour that are in one-to-one correspondence therewith; based on the first deformation magnitudes, determining a first grid deformation magnitude for each matching first model grid in the base 3D model associated with a respective first deformation control point, wherein the first model grid is used to describe the structural features of the target portion; and determining the first model deformation information of the base 3D model based on both the first deformation magnitudes and the first grid deformation magnitudes.

For example, the base 3D model includes multiple first model grids, meaning that these first model grids collectively form the base 3D model. Each first deformation control point matches at least one first model grid on the base 3D model. During the adjustment of the positions of the first deformation control points based on the first deformation magnitudes, controlling the deformation of the first model grids of the base 3D model based on their respective first grid deformation magnitudes enables the structural features of the target portion in the cross-section of the deformed base model to achieve size matching with those in the 2D ultrasound image. Consequently, a more accurate and realistic target 3D model of the patient's target portion can be obtained. Here, the first grid deformation magnitude is defined as the product of the corresponding first deformation magnitude and a predefined weight.

Step S106: based at least on the first model deformation information, deforming the base 3D model to obtain a target 3D model.

Exemplarily, based on the first model deformation information and using the first deformation reference points as benchmarks, the multiple first deformation control points on the base model section are deformed to (e.g. displaced onto) the first deformation reference point on the first target contour in the 2D ultrasound image. This deformation causes the first deformation control points to coincide with their corresponding first deformation reference points, resulting in substantial alignment between: (i) the second target contour corresponding to the structural features of the target portion on the base model section, and (ii) the first target contour corresponding to the structural features of the target portion on the 2D ultrasound image. By utilizing at least one frame of the first target contour of the target portion in the 2D ultrasound image as a deformation reference for the base 3D model of the target portion, a more accurate and realistic target 3D model of the target portion can be obtained.

In some embodiments, deforming the base 3D model to obtain the target 3D model based at least on the first model deformation information includes: performing positional alignment of the first deformation control points in the base 3D model toward the first deformation reference points serving as a first alignment target, until the first deformation control points achieve point-to-point coincidence with their corresponding first deformation reference points, thereby obtaining the target 3D model.

For example, taking a one-to-one correspondence between the first deformation reference points and the first deformation control points as an illustration, a one-to-one correspondence exists between: (i) the first deformation reference points on the first target contour corresponding to the structural features of the target portion in a frame of 2D ultrasound image, and (ii) the first deformation control points on the second target contour corresponding to the structural features of the target portion on the base model section of the base 3D model. Based on the correspondence between each first deformation reference point and its corresponding matched first deformation control point, multiple first deformation magnitudes of the base 3D model on the corresponding base model section are determined, thereby obtaining the first model deformation information of the base 3D model.

After the first model deformation information of the base 3D model is obtained, the positions of the first deformation control points are aligned to the positions of the first deformation reference points as an alignment target. During the alignment process, the positions of the corresponding first deformation control points are adjusted using the first deformation magnitudes, deforming the base model section of the base 3D model until each first deformation reference point coincides point-to-point with its corresponding first deformation control point, such that the structural features of the target portion on the base model section of the base 3D model match the structural features of the target portion in the 2D ultrasound image in size and morphology. This results in a more accurate and realistic target 3D model of the target portion.

It can be understood that the structural features of the target portion on the first target contour in a frame of 2D ultrasound image enable relatively precise adjustment to the contour of the target portion on a single section of the base 3D model. Therefore, the structural features of the target portion on the first target contours across multiple frames of ultrasound images obtained from different angles enable relatively precise adjustments to the contours of the target portion on multiple sections of the base 3D model. This enhances the accuracy of the resultant 3D model of the target portion and making it closer to the 3D model of the target portion in the 2D ultrasound image.

As shown in FIG. 7, the first target contour contains 18 first deformation reference points numbered 1a to 18a, while the second target contour contains 18 first deformation control points numbered 1b to 18b. In this configuration, a one-to-one correspondence is established between these points such that point 1a corresponds to point 1b, point 2a corresponds to point 2b, point 3a corresponds to point 3b, and so forth, continuing sequentially to point 17a corresponding to point 17b and point 18a corresponding to point 18b. Each pair of corresponding points has an associated first deformation magnitude.

After determining the corresponding first deformation magnitudes, the positions of the first deformation control points are adjusted based on the first deformation reference points, so that each first deformation control points undergo its corresponding first deformation magnitude relative to its paired first deformation reference points. Consequently, the paired points (the first deformation reference point 1a and the first deformation control point 1b, the first deformation reference point 2a and the first deformation control point 2b, the first deformation reference point 3a and the first deformation control point 3b, . . . , the first deformation reference point 17a and the first deformation control point 17b, and the first deformation reference point 18a and the first deformation control point 18b) coincide respectively. This process enables the structural features of the target portion on the base model section to match the size and morphology of the structural features of the target portion in the 2D ultrasound image, yielding a more accurate and realistic 3D model of the target portion.

Furthermore, by using the first target contour corresponding to the structural features of the target portion across multiple frames of 2D ultrasound images to adjust the second target contour corresponding to the structural features of the target portion on the base model section in the base 3D model, the obtained 3D model of the target portion can be made even more accurate and realistic.

In some embodiments, deforming the base 3D model based at least on the first model deformation information to obtain the target 3D model includes:
based on the first model deformation information, performing positional alignment of the first deformation control point in the base 3D model with the first deformation reference points serving as the first alignment target, and deforming the first model grid matching with the first deformation control points according to the first grid deformation magnitude, until the first deformation control points achieve point-to-point coincidence with their corresponding first deformation reference points, thereby obtaining the target 3D model.

As shown in FIG. 7, for instance, there are 18 first deformation reference points (numbered 1a to 18a respectively) on the first target contour, and there are also 18 first deformation control points (numbered 1b to 18b respectively) on the second target contour. In this configuration, a one-to-one correspondence is established between these points such that point 1a corresponds to point 1b, point 2a corresponds to point 2b, point 3a corresponds to point 3b, and so forth, continuing sequentially to point 17a corresponding to point 17b and point 18a corresponding to point 18b. Each pair of corresponding points has an associated first deformation magnitude.

The base 3D model includes multiple first model grids, each corresponding to at least one first deformation control point. In this embodiment, for clarity of explanation, it is assumed that each first model grid corresponds to one first deformation control point. For illustration purposes, the base 3D model includes 18 first model grids, specifically labeled 1c to 18c. The first deformation control point 1b corresponds to the first model grid 1c, the first deformation control point 2b corresponds to the first model grid 2c, the first deformation control point 3b corresponds to the first model grid 3c, and so forth. The first deformation control point 17b corresponds to the first model grid 17c, and the first deformation control point 18b corresponds to the first model grid 18c. In FIG. 7, only the first model grids 1c to 9c on the front side of the base 3D model are shown, whereas the first model grids 10c to 18c on the back side are not illustrated in the figure.

After determining the first deformation magnitudes corresponding to each first deformation control point, the first deformation control points are deformed based on the first deformation magnitudes, causing spatial coincidence between the first deformation reference point 1a and the first deformation control point 1b, between the first deformation reference point 2a and the first deformation control point 2b, between the first deformation reference point 3a and the first deformation control point 3b, with this correspondence pattern continuing through the spatial coincidence between the first deformation reference point 17a and the first deformation control point 17b, and finally between the first deformation reference point 18a and the first deformation control point 18b.

At the same time, the first model grid 1c of the base 3D model follows the deformation of the first deformation control point 1b based on the first grid deformation magnitude, until spatial coincidence is achieved between the corresponding first deformation control point 1b and the first deformation reference point 1a. The first grid deformation magnitude is defined as the product of the corresponding first deformation magnitude and a predefined weighting factor. After deformation, the structural features of the target portion in the base model section exhibit size-matching correspondence with the structural features of the target portion in the 2D ultrasound image. Thereby, a more accurate and realistic 3D model of the target portion of the patient can be obtained.

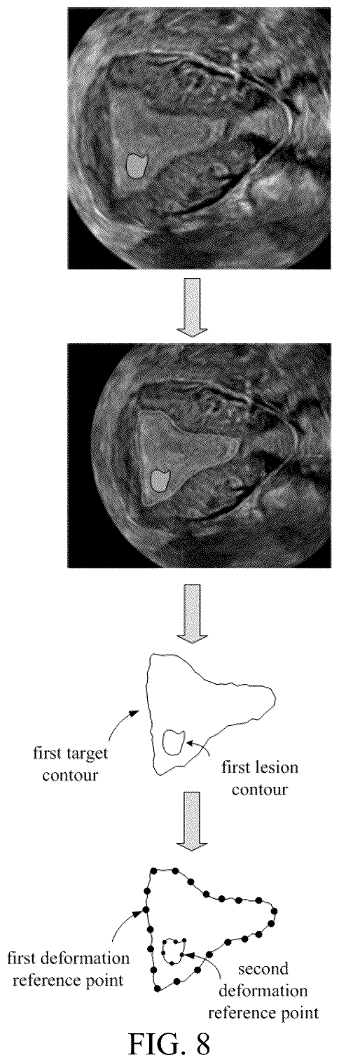

With reference to FIGS. 8 to 10, in some embodiments, the 2D ultrasound image at least includes the structural features and lesion features of the target portion. Correspondingly, the base model section includes the structural features and lesion features of the target portion, wherein the lesion features serve to indicate presence/absence of a lesion in the target portion. If there is a lesion, it can assist in diagnosing the type and/or location of the lesion.

In step S106, before deforming the base 3D model at least according to the first model deformation information to obtain the target 3D model, the method further includes:
obtaining the first lesion contour corresponding to the lesion features of the target portion in the 2D ultrasound image, and obtaining the second lesion contour corresponding to the lesion features of the target portion in the base model section;
determining a plurality of second deformation reference points on the first lesion contour, and determining, on the second lesion contour, at least two second deformation control points corresponding to at least two of the plurality of second deformation reference points; and
determining the second model deformation information of the base 3D model according to the second deformation reference points and the second deformation control points;
wherein, in step S106, deforming the base 3D model at least according to the first model deformation information to obtain the target 3D model includes:
deforming the base 3D model according to the first model deformation information and the second model deformation information to obtain the target 3D model.

In the present embodiment, acquisition of the first model deformation information may refer to the relevant description provided above, and thus is not repeated herein.

It is to be understood that the first lesion contour corresponding to the lesion features of the target portion in the 2D ultrasound image may be obtained either by manual delineation or by recognition using a model or algorithm.

As shown in FIG. 8, in some embodiments, obtaining the first lesion contour corresponding to the lesion features of the target portion in the 2D ultrasound image includes: in response to a second contour delineation operation performed by a user on the 2D ultrasound image, determining the first lesion contour corresponding to the lesion features of the target portion from the 2D ultrasound image.

By way of example, the obtained 2D ultrasound image of the target portion may be displayed on a display. A user, based on experience, can identify the structural contour corresponding to the structural features of the target portion from the 2D ultrasound image displayed on the display, and then, by manipulating an input device, perform a second contour delineation operation on the 2D ultrasound image, thereby delineating the first lesion contour corresponding to the lesion features of the target portion from the 2D ultrasound image through the second contour delineation operation. Thereby, the ultrasound imaging system 100 can, in response to the second contour delineation operation performed by the user on the 2D ultrasound image, determine the first lesion contour corresponding to the lesion features of the target portion from the 2D ultrasound image. The input device includes, but is not limited to, at least one of a keyboard, a mouse, a touch panel, a stylus, and a button.

In some embodiments, obtaining the first lesion contour corresponding to the lesion features of the target portion in the 2D ultrasound image includes: determining multiple second contour control points on the 2D ultrasound image, forming a second contour curve by interpolation between each pair of adjacent second contour control points, and defining said second contour curve as the first lesion contour corresponding to the lesion features of the target portion.

For example, the 2D ultrasound image of the target portion obtained can be displayed on a display; and a user can identify the lesion contour corresponding to the lesion features of the target portion from the 2D ultrasound image displayed on the display based on experience, and determine multiple second contour control points in the lesion contour corresponding to the lesion features of the target portion through an input device. After the ultrasound imaging system 100 detects the multiple second contour control points determined by the user, it forms a second contour curve by interpolation between each pair of adjacent second contour control points through a predefined interpolation algorithm, and designates the second contour curve as the first lesion contour corresponding to the lesion features of the target portion. It can be understood that the interpolation algorithm includes, but is not limited to, the B-spline interpolation algorithm (also known as the B-Spline interpolation method).

In some embodiments, the first lesion contour corresponding to the lesion features of the target portion in the 2D ultrasound image is obtained via a predefined second contour recognition model.

For example, the pre-trained second contour recognition model is used to perform contour recognition on the 2D ultrasound image to obtain the first lesion contour corresponding to the lesion features of the target portion. The second contour recognition model can be trained with 2D ultrasound images showing lesion features of the target portion as training data.

In some embodiments, after the first lesion contour is obtained, the plurality of second deformation reference points on the first lesion contour can be determined in accordance with a predefined third rule.

For example, the third rule may be: (1) setting a random number or a predefined number of second deformation reference points on the first lesion contour, with the distance between any two adjacent second deformation reference points being identical; or, (2) setting a random number or a predefined number of second deformation reference points on the first lesion contour, with the distance between any two adjacent second deformation reference points varying randomly; or, (3) setting a predefined number of second deformation reference points on the first lesion contour, with the distribution of the distance between any two adjacent second deformation reference points being associated with the target portion and/or the type of lesion.

In some embodiments, after the first lesion contour is obtained, determining the plurality of second deformation reference points on the first lesion contour comprises: determining a plurality of second key reference points on the first lesion contour; based on a predefined fourth rule, determining key contour points on a contour between each adjacent pair of the second key reference points, and taking both the second key contour points and the second key reference points as the plurality of second deformation reference points on the first lesion contour.

For example, the user sets multiple second key reference points on the first lesion contour based on experience; or, the ultrasound imaging system 100 automatically generates multiple second key reference points using a predefined deep learning algorithm or deep learning model, and then uses the second key reference points to segment the first lesion contour corresponding to the lesion features of the target portion into multiple second sub-contours, and then sets multiple second key contour points on each second sub-contour, thereby considering both the second key contour points and the second key reference points as multiple second deformation reference points on the first lesion contour. Optionally, the distance between two adjacent second key contour points on each second sub-contour is equal.

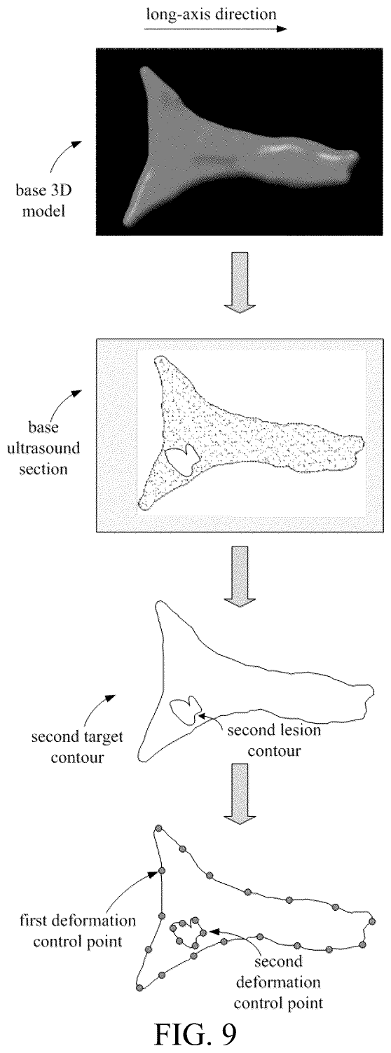

As shown in FIG. 9, in some embodiments, the second lesion contour corresponding to the lesion features of the target portion in the base model section is obtained, and at least two second deformation control points corresponding to at least two of the plurality of second deformation reference points are determined at least on the second lesion contour.

For example, the second lesion contour corresponding to the lesion features of the target portion in the base model section may be obtained: (1) by manually delineating the contour of the target contour (e.g., the lesion contour) corresponding to the base model section; (2) by using a pre-trained contour recognition model, inputting the base model section into the contour recognition model to obtain the corresponding contour in the base model section; (3) by identifying the corresponding contour in the base model section through a predefined contour recognition algorithm; or (4) by identifying the corresponding contour in the base model section through an edge recognition algorithm.

After obtaining the second lesion contour corresponding to the lesion features of the target portion, multiple second deformation control points are determined on the second lesion contour. The second deformation control points may be determined by manual selection by a user, or automatically generated on the second lesion contour by a predefined algorithm.

Optionally, the second lesion contour corresponding to the lesion features of the target portion in the base model section may be obtained by: in response to a fourth contour delineation operation performed by a user on the base model section, determining the second lesion contour corresponding to the lesion features of the target portion from the base model section.

For example, the obtained base model section of the target portion may be displayed on the display. Based on experience, the user identifies the lesion contour corresponding to the lesion features of the target portion from the base model section displayed on the display. The user then operates the input device to perform a fourth contour delineation operation on the base model section, thereby delineating the second lesion contour corresponding to the lesion features of the target portion from the base model section through the fourth contour delineation operation. In response to the user's fourth contour delineation operation on the base model section, the ultrasound imaging system 100 determines the second lesion contour corresponding to the lesion features of the target portion from the base model section.

Optionally, the second lesion contour corresponding to the lesion features of the target portion in the base model section may be obtained by: determining multiple fourth contour control points on the base model section, forming a fourth contour curve between each pair of adjacent fourth contour control points through interpolation (also known as interpolation algorithm), and taking the fourth contour curve as the second lesion contour corresponding to the lesion features of the target portion.

For example, the obtained base model section of the target portion may be displayed on the display; based on experience, the user can identify the lesion contour corresponding to the lesion features of the target portion from the base model section displayed on the display, and determine multiple fourth contour control points in the structural contour corresponding to the structural features of the target portion through the input device. After detecting the multiple fourth contour control points determined by the user, the ultrasound imaging system 100 forms a fourth contour curve between each pair of adjacent fourth contour control points through interpolation using a predefined interpolation algorithm, and takes the fourth contour curve as the second lesion contour corresponding to the lesion features of the target portion. It can be understood that the interpolation algorithm includes but is not limited to the B-spline interpolation algorithm, also known as the B-Spline interpolation method.

Optionally, the second lesion contour corresponding to the lesion features of the target portion in the base model section may be obtained by applying a predefined fourth contour recognition model.

For example, the fourth contour recognition model is pre-trained and used to perform contour recognition on the base model section, thereby obtaining the second lesion contour corresponding to the lesion features of the target portion. The fourth contour recognition model can be trained using images with the lesion features of the target portion as training data.

Optionally, the second lesion contour corresponding to the lesion features of the target portion in the base model section may be obtained by identifying edges of the lesion features of the target portion in the base model section using a predefined edge recognition algorithm.

After the second lesion contour corresponding to the lesion features of the target portion in the base model section is obtained, at least two second deformation control points corresponding to at least two of the multiple second deformation reference points are determined on the second lesion contour. Optionally, the second deformation control points on the second lesion contour and the second deformation reference points on the first lesion contour correspond one-to-one.

During deformation of the lesion features in the base 3D model, only a subset of the second deformation control points may require deformation. To ensure deformation quality, at least two second deformation control points corresponding to at least two of the multiple second deformation reference points are determined on the second lesion contour.

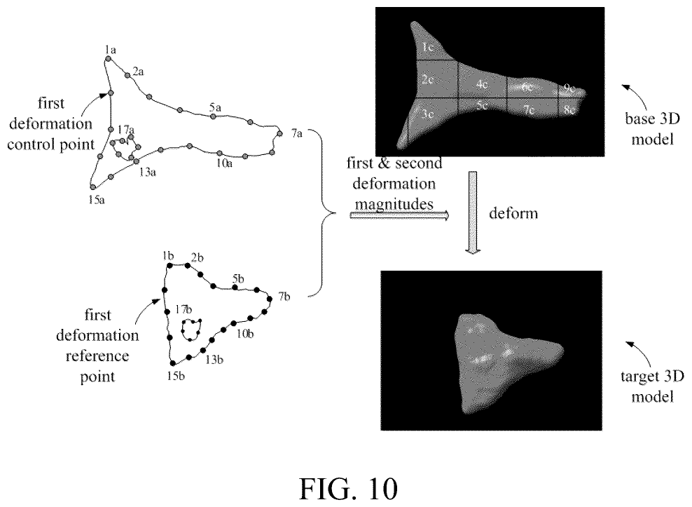

As shown in FIG. 10, after the second deformation control points corresponding to the second deformation reference points are determined on the second lesion contour, a second mapping relationship (also known as second deformation magnitude) between: (i) the second deformation control points on the second lesion contour, and (ii) the second deformation reference points on the first lesion contour may be obtained. Alternatively: (a) one second deformation control point on the second lesion contour corresponds to several second deformation reference points on the first lesion contour; or (b) several second deformation control points on the second lesion contour correspond to one second deformation reference point on the first lesion contour; wherein said second mapping relationship is obtained in either case. Thereby, the second model deformation information of the base 3D model may be obtained from the plurality of second deformation magnitudes generated by mapping relationships between: (i) the plurality of second deformation reference points, and (ii) the plurality of second deformation control points.

In some embodiments, after the first model deformation information and the second model deformation information are obtained, the process of deforming the base 3D model according to the first model deformation information and the second model deformation information to obtain the target 3D model includes:

deforming the structural features of the target portion in the base 3D model according to the first model deformation information, and deforming the lesion features of the target portion in the base 3D model according to the second model deformation information, thereby obtaining the target 3D model.

For example, based on the first model deformation information, it is determined to use at least the first deformation reference points as the first alignment target for performing position alignment on the first deformation control points of the base 3D model, until the first deformation control points coincide with the corresponding first deformation reference points; and based on the second model deformation information, it is determined to use at least the second deformation reference points as the second alignment target for performing position alignment on the second deformation control points of the base 3D model, until the second deformation control points coincide with the corresponding second deformation reference points, thereby obtaining the target 3D model.

Alternatively, based on the first model deformation information, it is determined to use at least the first deformation reference point as the first alignment target for performing position alignment on the first deformation control points of the base 3D model, while deforming the first model grid matched with the first deformation control points according to the first grid deformation magnitude, until the first deformation control points coincide with the corresponding first deformation reference points; and based on the second model deformation information, it is determined to use at least the second deformation reference point as the second alignment target for performing position alignment on the second deformation control points of the base 3D model, until the second deformation control points coincide with the corresponding second deformation reference points, thereby obtaining the target.

That is, in some embodiments, the method for generating a 3D ultrasound model includes steps S101 to S106. Specifically,
Step S101: obtaining a 2D ultrasound image corresponding to a target ultrasound section of a target portion, where the 2D ultrasound image includes at least the structural features and lesion features of the target portion;
Step S102: obtaining the first target contour corresponding to the structural features of the target portion and the first lesion contour corresponding to the lesion features of the target portion in the 2D ultrasound image, and determining multiple first deformation reference points on the first target contour and multiple second deformation reference points on the first lesion contour;
Step S103: obtaining a base 3D model corresponding to the target portion, and determining a base model section matching the target ultrasound section in the base 3D model, where the base model section includes the structural features and lesion features of the target portion;
Step S104: obtaining the second target contour corresponding to the structural features of the target portion in the base model section, and determining at least two first deformation control points corresponding to at least two of the multiple first deformation reference points on the second target contour; and obtaining the second lesion contour corresponding to the lesion features of the target portion in the base model section, and determining at least two second deformation control points corresponding to at least two of the multiple second deformation reference points on the second lesion contour;
Step S105: determining the first model deformation information of the base 3D model according to the first deformation reference points and the first deformation control points, and determining the second model deformation information of the base 3D model according to the second deformation reference points and the second deformation control points; and
Step S106: deforming the base 3D model according to the first model deformation information and the second model deformation information, thereby obtaining the target.

In some embodiments, the 2D ultrasound image also includes lesion features of the target portion; and before deforming the base 3D model to obtain the target 3D model at least based on the first model deformation information, the method further includes:
obtaining the lesion features of the target portion in the 2D ultrasound image; and
wherein, in step S106, deforming the base 3D model at least based on the first model deformation information to obtain the target 3D model, includes:
deforming the base 3D model based on the first model deformation information, and displaying the lesion representation on the deformed base 3D model according to the lesion feature to obtain the target 3D model.

The present embodiment differs from the preceding ones in that the database stores lesion data corresponding to lesion features, where the lesion data includes but is not limited to 3D lesion data and lesion images. After deforming the base 3D model using the first model deformation information: (1) corresponding lesion data is retrieved from the database according to the lesion features; (2) a lesion display position is determined on the deformed base 3D model according to the lesion features; and (3) the lesion data is displayed at the lesion display position—e.g., projecting the lesion image onto the model grid corresponding to the lesion display position is the deformed base 3D model, or displaying the 3D lesion data (such as a 3D lesion model) at the lesion display position on the deformed base 3D model.

Alternatively, the process of displaying the lesion representation on the deformed base 3D model according to the lesion feature to obtain the target 3D model includes: based on the lesion features, determining a 3D lesion morphology corresponding to the lesion region of the target portion, as well as a lesion mapping position of the lesion region on the deformed base 3D model; and displaying the 3D lesion morphology at the lesion mapping position on the deformed base 3D model to obtain the target 3D model.

In some embodiments, the 2D ultrasound image also includes lesion features of the target portion; and before deforming the base 3D model to obtain the target 3D model at least based on the first model deformation information, the method further includes:
obtaining the lesion features of the target portion in the 2D ultrasound image; and
wherein, deforming the base 3D model at least based on the first model deformation information to obtain the target 3D model includes:
deforming the structural features of the target portion in the base 3D model based on the first model deformation information;
determining the first lesion contour corresponding to the lesion region of the target portion, as well as the lesion mapping position of the lesion region on the deformed base 3D model based on the lesion features of the target portion; and
generating the 3D lesion morphology corresponding to the lesion region by interpolating the first lesion contour, and displaying the 3D lesion morphology at the lesion mapping position on the deformed base 3D model, thereby obtaining the target.

Optionally, the first deformation reference points and the first deformation control points correspond one-to-one; and the step of deforming the structural features of the target portion in the base 3D model according to the first model deformation information includes:
determining to use at least the first deformation reference points as a first alignment target, and performing positional alignment on the first deformation control points in the base 3D model according to the first model deformation information, until the first deformation control points coincide with their corresponding first deformation reference points.

In some embodiments, the method further includes: (a) determining a first target position using the target contour in the 2D ultrasound image and generating a measurement marker; (b) performing measurement at the first target position with the measurement marker to obtain measurement data; (c) determining a second target position on the target 3D model that corresponds to the first target position; and (d) associatively displaying the measurement marker and measurement data at the second target position, wherein the target contour includes the first target contour and/or the first lesion contour.

For example, when dimension measurement (e.g., length, width) at a predefined position on the first target contour in a 2D ultrasound image is required: (a) a corresponding measurement marker is generated at a to-be-measured position in response to an input signal generated by manipulating the input device by a user; (b) dimensional measurement is performed via the measurement marker at the predefined position to obtain corresponding measurement data; and (c) the measurement data is displayed at a corresponding second position in the target 3D model. Here, both the first target position and the second target position are used to represent the same position of the target portion in 2D and 3D spaces.

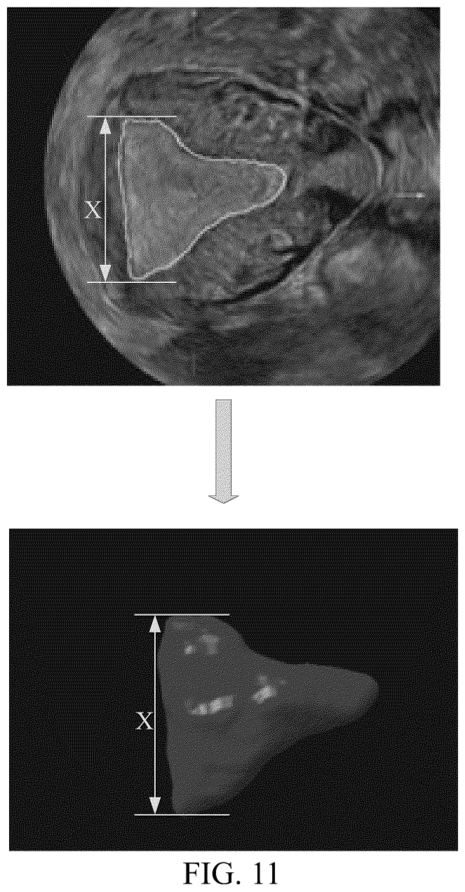

As shown in FIG. 11, in the 2D ultrasound image, the width at the predefined position on the first target contour corresponding to the target portion is measured using the measurement marker to obtain the measurement data X. In the 3D model corresponding to the target portion, the measurement data X is displayed at the second position which is the same as the first position, so that the user can more intuitively obtain the corresponding size of the target portion from the 3D model.

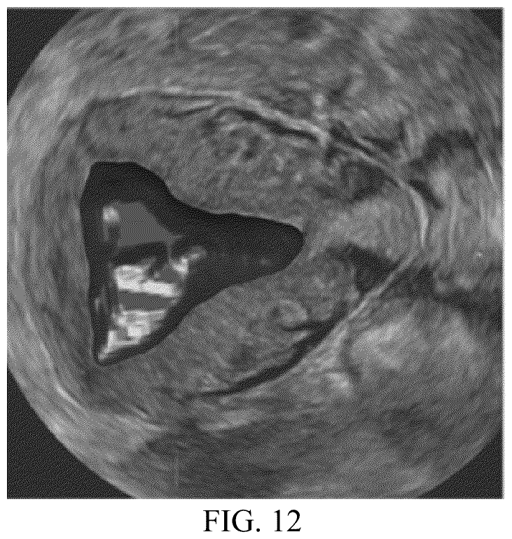

With reference to FIG. 12, in some embodiments, the method further includes: simultaneously displaying or displaying in fusion at least one frame of 2D ultrasound image of the target portion and the target 3D model in the same display interface.

For example, the display methods for the 2D ultrasound image and the 3D model corresponding to the target portion may include: displaying them respectively on different display screens; or, displaying them respectively in different display interfaces of the same display; or, displaying them in different display windows within the same display interface; or, displaying them in different display areas of the same display interface; or, performing fused display of both in the same display interface.

Optionally, displaying in fusion at least one frame of 2D ultrasound image and the target 3D model of the target portion in the same display interface includes:
displaying at least one frame of 2D ultrasound image and the target 3D model of the target portion in the same display interface, wherein the target 3D model covers the target portion where the structural features of the target portion are located in the 2D ultrasound image.

As shown in FIG. 12, the edges of the target 3D model approximately coincide with the edges of the structural features of the target portion in the 2D ultrasound image.

It should be understood that the color-coding of certain structural elements in the drawings accompanying this specification serves merely to visually distinguish between different structures and to facilitate understanding of the technical solution claimed in the present application in conjunction with the textual description. No protection is claimed for the specific colors shown in the drawings, and these colors may be varied as necessary.

It is to be understood that the term “and/or” as used in the specification and appended claims of this application designates any combination comprising one or more of the associated listed items and encompasses all possible combinations, including said combinations. Additionally, the terms “comprise”, “include” or any variations thereof in this document are intended to specify non-exclusive inclusion, such that a process, method, article, or system comprising a series of elements includes not only those explicitly recited elements, but also other elements not expressly listed or inherent to such process, method, article, or system. Further, in the absence of additional limitations, an element defined by the phrase “comprising one . . . ” does not preclude the presence of additional identical elements in the process, method, article, or system that includes said element.

The numbering of embodiments in the foregoing description serves descriptive purposes only and does not denote any preference among the embodiments. While the above represents specific embodiments of the present application, the scope of protection is not limited thereto. Those skilled in the art may readily contemplate various equivalent modifications or substitutions within the technical scope disclosed herein, all of which shall fall within the protection scope of this application. Accordingly, the protection scope of this application shall be defined by the appended claims.

What is claimed is:

1. A method for generating a three-dimensional ultrasound model, comprising:
obtaining a two-dimensional ultrasound image corresponding to a target ultrasound section of a target portion, wherein the two-dimensional ultrasound image contains structural features of the target portion and lesion features of the target portion;
obtaining a first target contour corresponding to the structural features of the target portion and a first lesion contour corresponding to the lesion features of the target portion in the two-dimensional ultrasound image, and determining a plurality of first deformation reference points on the first target contour and a plurality of second deformation reference points on the first lesion contour;
obtaining a base three-dimensional model corresponding to the target portion, and determining a base model section matching the target ultrasound section in the base three-dimensional model, wherein the base model section contains the structural features of the target portion and the lesion features of the target portion;
obtaining a second target contour corresponding to the structural features of the target portion in the base model section, and determining on the second target contour at least two first deformation control points corresponding to at least two of the plurality of first deformation reference points;
obtaining a second lesion contour corresponding to the lesion features of the target portion in the base model section, and determining on the second lesion contour at least two second deformation control points corresponding to at least two of the plurality of second deformation reference points;
determining first model deformation information of the base three-dimensional model according to the first deformation reference points and the first deformation control points,
determining second model deformation information of the base three-dimensional model according to the second deformation reference points and the second deformation control points; and
deforming the structural features of the target portion in the base three-dimensional model according to the first model deformation information, and deforming the lesion features of the target portion in the base three-dimensional model based on the second model deformation information, thereby obtaining the target three-dimensional model.

2. The method for generating a three-dimensional ultrasound model according to claim 1, wherein obtaining the first target contour corresponding to the structural features of the target portion in the two-dimensional ultrasound image, comprises:
in response to a first contour delineation operation performed by a user on the two-dimensional ultrasound image, determining the first target contour corresponding to the structural features of the target portion from the two-dimensional ultrasound image;
or, determining a plurality of first contour control points on the two-dimensional ultrasound image, forming a first contour curve by interpolation between each pair of adjacent first contour control points, and defining the first contour curve as the first target contour corresponding to the structural features of the target portion;
or, obtaining the first target contour corresponding to the structural features of the target portion from the two-dimensional ultrasound image via a predefined first contour recognition model.

3. The method for generating a three-dimensional ultrasound model according to claim 1, wherein determining the plurality of first deformation reference points on the first target contour, comprises:
determining the plurality of first deformation reference points on the first target contour based on a predefined first rule;
or, determining a plurality of first key reference points on the first target contour, determining a first key contour point on a contour between each adjacent two of the first key reference points based on a predefined second rule, and defining the first key contour points and the first key reference points as the plurality of first deformation reference points on the first target contour;
and wherein determining the plurality of second deformation reference points on the first lesion contour, comprises:
determining the plurality of second deformation reference points on the first lesion contour based on a predefined third rule;
or, determining a plurality of second key reference points on the first lesion contour, and determining a second key contour point on a contour between each adjacent two of the second key reference points based on a predefined fourth rule, and defining the second key contour points and the second key reference points as the plurality of second deformation reference points on the first lesion contour.

4. The method for generating a three-dimensional ultrasound model according to claim 1, wherein obtaining the two-dimensional ultrasound image corresponding to the target ultrasound section of the target portion, comprises:
obtaining corresponding two-dimensional ultrasound images from at least two different long-axis planes of the target portion, wherein any two different long-axis planes intersect at an angle;
or, obtaining at least one two-dimensional ultrasound image from a long-axis plane of the target portion and at least one two-dimensional ultrasound image from a short-axis plane of the target portion.

5. The method for generating a three-dimensional ultrasound model according to claim 1, wherein determining the first model deformation information of the base three-dimensional model according to the first deformation reference points and the first deformation control points, comprises:
determining a plurality of corresponding first deformation magnitudes between the plurality of first deformation reference points on the first target contour and the plurality of first deformation control points matching said first deformation reference points on the second target contour, and determining the first model deformation information of the base three-dimensional model at least according to the plurality of first deformation magnitudes;
or, determining a plurality of corresponding first deformation magnitudes between the plurality of first deformation reference points on the first target contour and the plurality of first deformation control points corresponding one-to-one to said first deformation reference points on the second target contour, determining a first grid deformation magnitude corresponding to a first model grid matched to each first deformation control point in the base three-dimensional model based on the first deformation magnitudes, said first model grid being configured to describe the structural features of the target portion; and determining the first model deformation information of the base three-dimensional model according to the first deformation magnitudes and the first grid deformation magnitudes;
and wherein determining the second model deformation information of the base three-dimensional model according to the second deformation reference points and the second deformation control points, comprises:
determining a plurality of corresponding second deformation magnitudes between the plurality of second deformation reference points on the first lesion contour and the plurality of second deformation control points matching said second deformation reference points on the second lesion contour, and determining the second model deformation information of the base three-dimensional model according to the plurality of second deformation magnitudes;
or, determining a plurality of corresponding second deformation magnitudes between the plurality of second deformation reference points on the second lesion contour and the plurality of second deformation control points corresponding one-to-one to said second deformation reference points on the second lesion contour, determining a second grid deformation magnitude corresponding to a second model grid matched to each second deformation control point in the base three-dimensional model based on the second deformation magnitudes, said second model grid being configured to describe the lesion features of the target portion; and determining the second model deformation information of the base three-dimensional model according to the second deformation magnitudes and the second grid deformation magnitudes.

6. The method for generating a three-dimensional ultrasound model according to claim 1, wherein
the first deformation reference points correspond one-to-one with the first deformation control points,
the second deformation reference points correspond one-to-one with the second deformation control points, and
deforming the structural features of the target portion in the base three-dimensional model according to the first model deformation information, and deforming the lesion features of the target portion in the base three-dimensional model according to the second model deformation information, comprise:
performing positional alignment of the first deformation control points in the base three-dimensional model using at least the first deformation reference points as a first alignment target according to the first model deformation information, until the first deformation control points coincide with corresponding first deformation reference points; and performing positional alignment of the second deformation control points in the base three-dimensional model using at least the second deformation reference points as a second alignment target according to the second model deformation information, until the second deformation control points coincide with corresponding second deformation reference points, thereby obtaining the target three-dimensional model.

7. The method for generating a three-dimensional ultrasound model according to claim 1, further comprising:
determining a first target position based on a target contour of the two-dimensional ultrasound image and generating a measurement marker, and measuring the first target position based on the measurement marker to obtain measurement data; and
determining a second target position matched to the first target position on the target three-dimensional model, and displaying the measurement marker and measurement data in association at the second target position, wherein the target contour includes the first target contour and/or the first lesion contour.

8. A method for generating a three-dimensional ultrasound model, comprising:
obtaining a two-dimensional ultrasound image corresponding to the target ultrasound section of the target portion, wherein the two-dimensional ultrasound image at least contains structural features of the target portion;
obtaining a first target contour corresponding to the structural features of the target portion in the two-dimensional ultrasound image, and determining a plurality of first deformation reference points on the first target contour;
obtaining a base three-dimensional model corresponding to the target portion, and determining a base model section matching the target ultrasound section in the base three-dimensional model, wherein the base model section contains the structural features of the target portion;
obtaining a second target contour corresponding to the structural features of the target portion in the base model section, and determining on the second target contour at least two first deformation control points corresponding to at least two of the plurality of first deformation reference points;
determining first model deformation information of the base three-dimensional model according to the first deformation reference points and the first deformation control points; and
deforming the base three-dimensional model at least according to the first model deformation information to obtain a target three-dimensional model.

9. The method for generating a three-dimensional ultrasound model according to claim 8, wherein the first deformation reference points correspond one-to-one with the first deformation control points, and deforming the base three-dimensional model at least according to the first model deformation information to obtain the target three-dimensional model, comprises:
performing positional alignment of the first deformation control points in the base three-dimensional model using at least the first deformation reference points as a first alignment target according to the first model deformation information, until the first deformation control points coincide with corresponding first deformation reference points, thereby obtaining the target three-dimensional model.

10. The method for generating a three-dimensional ultrasound model according to claim 8, wherein obtaining the two-dimensional ultrasound image corresponding to the target ultrasound section of the target portion, comprises:
obtaining corresponding two-dimensional ultrasound images from at least two different long-axis planes of the target portion, wherein any two different long-axis planes intersect at an angle;
or, obtaining at least one two-dimensional ultrasound image from a long-axis plane of the target portion and at least one two-dimensional ultrasound image from a short-axis plane of the target portion.

11. The method for generating a three-dimensional ultrasound model according to claim 8, wherein obtaining the first target contour corresponding to the structural features of the target portion in the two-dimensional ultrasound image, comprises:
in response to a first contour delineation operation performed by a user on the two-dimensional ultrasound image, determining the first target contour corresponding to the structural features of the target portion from the two-dimensional ultrasound image;
or, determining a plurality of first contour control points on the two-dimensional ultrasound image, forming a first contour curve by interpolation between each pair of adjacent first contour control points, and defining the first contour curve as the first target contour corresponding to the structural features of the target portion;
or, obtaining the first target contour corresponding to the structural features of the target portion from the two-dimensional ultrasound image via a predefined first contour recognition model.

12. The method for generating a three-dimensional ultrasound model according to claim 8, wherein determining the plurality of first deformation reference points on the first target contour, comprises:
determining the plurality of first deformation reference points on the first target contour based on a predefined first rule;
or, determining a plurality of first key reference points on the first target contour, determining a first key contour point on a contour between each adjacent two of the first key reference points based on a predefined second rule, and defining the first key contour points and the first key reference points as the plurality of first deformation reference points on the first target contour.

13. The method for generating a three-dimensional ultrasound model according to claim 8, wherein the two-dimensional ultrasound image further contains lesion features of the target portion, and before deforming the base three-dimensional model at least according to the first model deformation information to obtain a target three-dimensional model, the method further comprises:
obtaining the lesion features of the target portion in the two-dimensional ultrasound image; and
wherein deforming the base three-dimensional model at least according to the first model deformation information to obtain the target three-dimensional model comprises:
deforming the base three-dimensional model according to the first model deformation information, and displaying a lesion representation on the deformed base three-dimensional model according to the lesion feature, thereby obtaining the target three-dimensional model.

14. The method for generating a three-dimensional ultrasound model according to claim 13, wherein displaying the lesion representation on the deformed base three-dimensional model according to the lesion features comprises:
according to the lesion feature, determining a three-dimensional lesion morphology corresponding to a lesion region in the target portion and a lesion mapping position of the lesion region on the deformed base three-dimensional model;
displaying the three-dimensional lesion morphology at the lesion mapping position on the deformed base three-dimensional model to obtain the target three-dimensional model.

15. The method for generating a three-dimensional ultrasound model according to claim 8, wherein the two-dimensional ultrasound image further contains lesion features of the target portion; and before deforming the base three-dimensional model at least according to the first model deformation information to obtain a target three-dimensional model, the method further comprises:
obtaining the lesion features of the target portion in the two-dimensional ultrasound image;
and wherein deforming the base three-dimensional model at least according to the first model deformation information to obtain the target three-dimensional model comprises:
deforming the structural features of the target portion in the base three-dimensional model according to the first model deformation information;
according to the lesion features of the target portion, determining a first lesion contour corresponding to the lesion region in the target portion and a lesion mapping position of the lesion region on the deformed base three-dimensional model; and
generating a three-dimensional lesion morphology corresponding to the lesion region by interpolating the first lesion contour and displaying the three-dimensional lesion morphology at the lesion mapping position on the deformed base three-dimensional model to obtain the target three-dimensional model.

16. The method for generating a three-dimensional ultrasound model according to claim 15, wherein the first deformation reference points correspond one-to-one with the first deformation control points, and deforming the structural features of the target portion in the base three-dimensional model according to the first model deformation information comprises:
performing positional alignment of the first deformation control point in the base three-dimensional model using at least the first deformation reference points as a first alignment target according to the first model deformation information, until the first deformation control points coincide with corresponding first deformation reference points.

17. The method for generating a three-dimensional ultrasound model according to claim 8, further comprising:
determining a first target position based on a target contour of the two-dimensional ultrasound image and generating a measurement marker, and measuring the first target position based on the measurement marker to obtain measurement data;
determining a second target position matched to the first target position on the target three-dimensional model, and displaying the measurement marker and the measurement data in association at the second target position, wherein the target contour comprises the first target contour and/or the first lesion contour.

18. The method for generating a three-dimensional ultrasound model according to claim 8, further comprising:
displaying simultaneously at least one frame of two-dimensional ultrasound image of the target portion and the target three-dimensional model in a same display interface;
or, fusing and displaying at least one frame of two-dimensional ultrasound image of the target portion and the target three-dimensional model in a same display interface.

19. The method for generating a three-dimensional ultrasound model according to claim 18, wherein fusing and displaying at least one frame of two-dimensional ultrasound image of the target portion and the target three-dimensional model in a same display interface comprises:
displaying at least one frame of two-dimensional ultrasound image of the target portion and the target three-dimensional model in the same display interface, wherein the target three-dimensional model covers a target region where the structural features of the target portion is located in the two-dimensional ultrasound image.

20. An ultrasound imaging system, comprising:
an ultrasound probe, comprising a plurality of transducer elements;
a transmit circuit, configured to excite the ultrasound probe to emit ultrasound waves to a target portion;
a receive circuit, configured to control the ultrasound probe to receive echo signals of the ultrasound waves;
a memory, configured to store a program;
a processor, configured to:
generate a two-dimensional ultrasound image corresponding to a target ultrasound section of the target portion according to the echo signals, wherein the two-dimensional ultrasound image at least contains structural features of the target portion;
obtain a first target contour corresponding to the structural features of the target portion in the two-dimensional ultrasound image, and determining a plurality of first deformation reference points on the first target contour;
obtain a base three-dimensional model corresponding to the target portion, and determining a base model section matching the target ultrasound section in the base three-dimensional model, wherein the base model section contains the structural features of the target portion;
obtain a second target contour corresponding to the structural features of the target portion in the base model section, and determining on the second target contour at least two first deformation control points corresponding to at least two of the plurality of first deformation reference points;
determine first model deformation information of the base three-dimensional model according to the first deformation reference points and the first deformation control points; and
deform the base three-dimensional model at least according to the first model deformation information to obtain a target three-dimensional model.
